(19) United States
(12) Patent Application Publication
(10) Pub. No.: US 2012/0232257 A1
(43) Pub. Date: Sep. 13, 2012

(54) Novel Method for Directly Nitration of OH-, SH-and NHR-Functions in Organic Molecules by Means of in Situ Generated Carbonic Acid Dinitrate

(75) Inventor: Nikolas Pietrzik, Illertissen (DE)

(73) Assignee: SYNOVO GMBH, Tubingen (DE)

(21) Appl. No.: 13/390,311

(22) PCT Filed: Oct. 4, 2010
(86) PCT No.: PCT/IB10/03061

§ 371 Date: Apr. 27, 2012

(30) Foreign Application Priority Data
Aug. 11, 2009 (DE) 10 2009 037 555.4

(51) Int. Cl. C07C 201/02; C07H 5/04; C07H 1/00; C07C 203/04; C07D 251/06; C08B 5/02; C07H 7/02

(52) U.S. Cl. 536/17.2; 536/35; 536/55; 558/485; 558/483; 544/215

(57) ABSTRACT

The invention relates to a nitration method having the following principles: a phosgene species is converted with two equivalent silver nitrates into a double-mixed anhydride of carbonic acid and nitric acid, known here as carbonic acid dinitrate (I). Said operation is carried out in situ, and the formed dinitrate decomposes spontaneously. In addition to carbon dioxide, nitrate ions and nitronium ions are formed, said ions comprising electrophiles which are necessary for nitration. The solution which is used is acetonitrile, and is insignificant if the alcohol species is dissolved or suspended. The necessary equivalent silver nitrates are introduced into the system and optionally heated or cooled to the desired temperature. Subsequently, the acid chloride is introduced slowly, drop by drop or slowly little by little. Phosgene, diphosgene, triphosgene and chloroformic acid ester can be used as carbonic acid dichloride and monochloride, and their thiocarbonic acid analogues. A brown colouration and precipitated silver chloride display the formation of the carbonic acid reactants, said brown colouration rapidly discolouring due to an immediate reaction of the nitronium ions with the substrate with is to be nitrated. Towards the end of the addition of phosgene, the brown colouration remains for longer and longer until it no longer disappears. Then, it is stirred for another hour at room temperature. In the event of high acid-sensitive educts, non-nucleophilic nitrogen bases such as DBU can be added to the system in order to intercept the formation of nitric acid.

### BACKGROUND OF INVENTION

The preparation of nitro compounds and nitric acid esters is an important and extensive field of application for the electrophilic substitution reaction. In general nitronium cations have to be supplied as electrophilic species, in most cases generated in situ. Some rare cases are published where they are presented as a salt, e.g. nitronium tetrafluoroborate for regioselective aromatic nitration. Conventional nitration techniques employ classic nitrosulfuric acids in differing compositions or mixed anhydrides of nitric acid with carboxylic acids, as e.g. acetyl nitrate or benzoyl nitrate. The disadvantages of these methods are obvious: Strong acidic conditions, oxidizing reactants, explosive hazards and complex temperature programs in addition to low (regio-) selectivity. Hence reagents suitable for this chemistry must be stable which excludes many substance classes from the very beginning, e.g. reducing sugars or unstable natural products. The same applies to working conditions and technical apparatus standards. Further drawbacks of mixed acid anhydrides are explosion hazards, a vast possibility of undesired side reactions such as acylations, which can even the majority of reaction yields in the case of OH nitration.

Herein an innovative procedure is presented, applying a novel, in situ and mildly accessible nitronium donor, making highly selective O-, S- and N-nitration possible. The donor is prepared when a phosgene species is reacted with silver nitrate to generate carbonic acid dinitrate. This again spontaneously decomposes, liberating the required electrophilic nitronium cations. Using this method, acid labile substrates such as reducing sugars, acetals or stereochemically demanding amino acids can be subject to selective nitration in a “one pot” procedure. Similarly pernitration of polyols can be achieved.

Nitro celluloses in differing degrees of nitration are a substance class of great technical interest. Their field of application ranges from polymer additiva to filter membranes, thermoplastics and energetic materials for combustion.

Up to date preparation methods employ nitrosulfuric acids which are cheap but offer only limited control of reaction with regards to stochiometry or side reactions (sulfonation, chain degradation etc.). Higher degree of nitration is achieved through prolonged reaction time, which also means that the cellulose is subject to an increased rate of chain degradation or other undesired side reactions. Hence the technically highest degree of nitration is 13.45% nitrate content which is below the theoretical maximum. The method described here allows higher degrees of nitration. The method also provides for improved safety standards, waste reduction, and ecological impact.

### DESCRIPTION

In situ generated carbonic acid dinitrate serves as a mild source of nitronium electrophiles which allows nitration of reducing as well as non-reducing mono, poly and oligosaccharides in short times. Degree of nitration is determined rather via reaction stochiometry rather than reaction times. One equivalent of carbonic acid dinitrate is consumed to derivatize one hydroxyl (or analogous) function. Reaction times vary but are typically rapid or near instantaneous. In case of highly acid labile substrates the reaction system may be buffered with certain sterically hindered bases to neutralise the slowly forming nitric acid. All reaction steps are carried out in organic media resulting in easy work up and quick drying times.

Due to the mildness of the procedure chain length of a polymer like cellulose or previously introduced functional groups remain untouched. As a consequence, novel nitro celluloses with altered material properties become accessible. Furthermore acid labile and readily oxidizable substrates such as acetals or thiols may be derivatized without first generating protective groups on the sensitive functional groups.

### Synthesis

The following principle is the basis of the present novel nitration process: A phosgene species is reacted with 2 equivalents of silver nitrate to yield the mixed anhydride of carbonic acid and nitric acid, called carbon dinitrate.

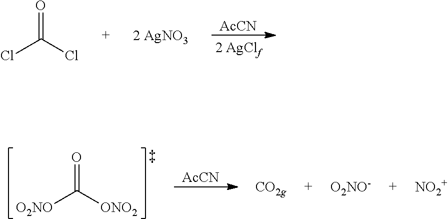

This is done in situ such that the dinitrate decomposes spontaneously, yielding gaseous carbon dioxide, nitrate and nitronium ions. The latter cations are the electrophilic species used for nitration. Acetonitrile serves as solvent as it readily dissolves silver nitrate, whereas it makes no difference if the target for nitration is also dissolved or rather suspended. After cooling or warming to desired reaction temperatures the required equivalents of acid chloride are added to the system. As mono- or dichlorides of carbonic acid one may use phosgene, diphosgene, triphosgene, chloroformic acid esters as well as their thio carbonic acid counterparts. Precipitating silver chloride and color change to brown (nitrous species) indicate formation of the reactive anhydride. Immediate decoloration is observed due to reaction of nitronium ions with the substrate. Speed of decoloration decreases to the end of reaction and the system remains brown in color if excess of nitrating agent is used. In case of highly acid labile reactants the system can also be buffered with non-nucleophilic bases (e.g. DBU) to scavange and neutralise newly formed nitric acid.

### SYNTHESIS EXAMPLES

### 1) Nitration of Cellulose

Cellulose is suspended in acetonitrile, the corresponding amount of silver nitrate is added while stirring. Subsequently the phosgene species is carefully added dropwise so that the system temperature does not exceed 45° C. After one hour of stirring the precipitating silver salt is filtered off and the filtrate is evaporated in vacuo. Crude products were taken up in specific solvents due to their solubility depending on degree of nitration (i.e. the higher the nitration grade the lower polarity required). In the case of collodium the product can be obtained as a film in variable thickness from acetone.

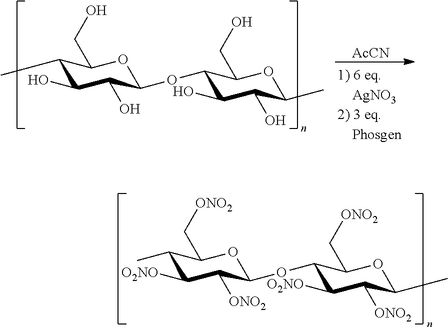

A specific degree of nitration can be achieved by choice of reaction stochiometry, with regards to the molar equivalents of silver nitrate/phosgene species. 4 equivalents of silver nitrate with 2 equivalents of phosgene yield collodium with a slightly increased nitration grade then a comparable commercial available product (Fluka 09986). It is examined by elemental analysis (apparatus: EuroEA elemental analyzer).

Fluka 09986 found: N, 11.19; C, 27.14; H, 3.42. quotient N/C: 0.42;

NP-NC-001 found: N, 12.91; C, 30.32; H, 3.06. quotient N/C: 0.43.

These findings in combination with a differing performance of said products when heated to high temperature (T>170° C., apparatus: Stuart Scientific SMP10) both point to differences in their macromolecular structure:

Colloidium Fluka 09986 ˜173° C.: decomposition, formation of nitrous vapors;

NP-NC-001 182-188° C.: melting; no sign of decomposition

Hence, this procedure is not only a means of nitro cellulose synthesis but may furthermore leads to a products with altered physical behavior—probably due to another macromolecular structure perhaps related to longer chain length or higher purity. This may lead to novel candidates for usage as e.g. thermoplastics, membrane materials or propellants.

### 2) Nitration of β-D-Glucose

Unprotected β-D-glucopyranoside is suspended in acetonitrile and silver nitrate is added while stirring. The mixture is cooled to 0° C. and the phosgene species is added dropwise. The system is allowed to warm up to ambient temperature while stirring for one further hour. Then silver salts are filtered off and the filtrate is evaporated in vacuo.

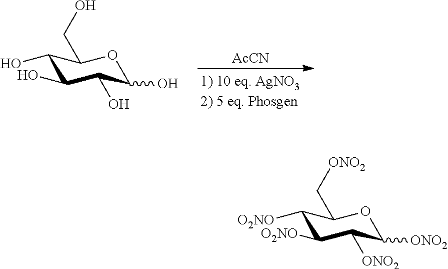

Pernitrated sugars or saccharides with a high degree of nitration must be considered explosive and were kept in the refrigerator as dichloromethane solutions.

As with the synthesis of nitro cellulose the degree of nitration can be appointed by choice of molar ratio (substrate/silver nitrate/carbon dinitrate) in this example as well. Dinitration is achieved when 2 equivalents of phosgene and 4 equivalents of silver nitrate are employed at 0° C. According to thin layer chromatography one defined product is obtained. The products exact stereochemistry is not yet determined in this example. Degree of nitration is determined via elemental analysis (apparature: EuroEA elemental analyzer).

calculated (dinitration, 1/5 DCM, 1/5 H₂O): C, 25.61; H, 3.74; N, 9.64.

found (NP-03-NGlc, 1/5 DCM, 1/5 H₂O): C, 24.94; H, 3.47; N, 10.20.

Anomer nitrates may function as valuable starting material for glycosylation reactions, especially when accessible without much effort regarding protective group manipulation etc. This method makes pernitrated glycopyranosides available so far not known to literature, starting from reducing sugars. These products can be seen as members of the “polynitrated Glycosides” (PNGs) explosive class.

### 3) Nitration of methyl-4,6-O-benzyliden-α-D-Glucopyranosid

The following example shows the procedure's mildness and selectivity. Methyl-4,6-O-benzylidene-α-D-glucopyranoside is subject to the process. Therefore, it is dissolved in acetonitrile together with 4 equivalents of silver nitrate and the clear solution is cooled to 0° C. in an ice bath. 2 equivalents of phosgene are slowly added and the system is allowed to warm up to room temperature. Stirring is continued for half an hour and the reaction progress is determined with thin layer chromatography (ethyl acetate-cyclohexane 1:1). After consumption of starting materials silver salts are filtered off, ethyl acetate is added to the filtrate and the organic solution is washed with water (3×). After separation the organic phase is dried over sodium sulfate, filtrated and evaporated in vacuo. Column chromatography (ethyl acetate-cyclohexane 1:3) of the crude products furnished the dinitrated sugar with traces of mononitrated species.

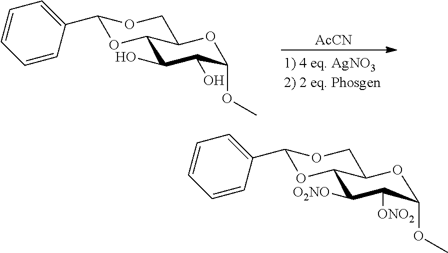

NMR spectroscopy (apparature: Bruker Avance400 Ultrashield) clearly shows that solely O-nitration was achieved. Under the described conditions no aromatic substitution (S_{Ae}) was observed. The process is also orthogonal to the applied protective group strategy, the benzylidene acetal remained intact. This reaction is not possible using existing nitration procedures and makes the remarkable selectivity of the process evident.

The characteristic signals of the aromatic carbon atoms of the benzylidene acetal clearly indicate the unsubstituted presence of this group after the reaction. Neither loss nor nitration of the aromatic system occurred.

| Chemical shifts (ppm): | signal allocation: |
|---|---|
| 136.4 | 1 C, quarternary, viccinal to acetal carbon atom, |
| 129.4 | 2 C, tertiary, ortho-position, unsubstituted, |
| 128.4 | 2 C, tertiary, meta-position, unsubstituted, |
| 126.1 | 1 C, tertiary, para-position, unsubstituted. |

### 4) Nitration of Pentaerythritol Derivatives and N-Acetylcystein (ACC)

Molecules with nitrothio or nitrosothio functions are substrates of certain medicinal relevance, as they might serve as NO donors or modulators in living organisms. Furthermore combustible behavior of O-nitro compounds (e.g. nitro glycerin or nitropenta) can be altered if hydroxyl groups are exchanged with thiol groups, hence application to SH-nitration. Therefore the following two examples were applied.

Thiols were dissolved in acetonitrile along with 2 equivalents of silver nitrate per SH and/or OH group and the clear solutions were cooled to 0° C. One equivalent per SH and/or OH group of phosgene is slowly added and the systems were allowed to warm up to ambient temperature. Stirring is continued for one hour and the reaction determined with thin layer chromatography (ethyl acetate-cyclohexane 1:1, in case of ACC methanol-chloroform 1:3). After consumption of starting materials silver salts are filtered off, ethyl acetate is added to the filtrate and the organic solution is washed with water (3×). After separation the organic phase is dried over sodium sulfate, filtrated and evaporated in vacuo. Column chromatography (as with thin layer chromatography) of the crude products furnished the desired products.

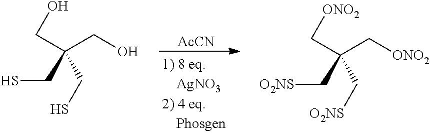

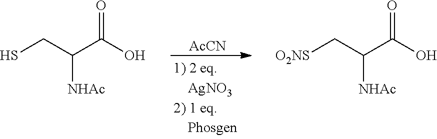

The experiments indicated that nitration of the sulfur species was achieved instead of oxidation, which is an undesired side reaction of the classical nitration conditions. Vibrational spectroscopy investigation of the products (IR, apparatus: Bruker Tensor 27) makes SH nitration evident as spectral data show the vanishing of the characteristic signal for the thiol group of the starting materials at ν˜2550.
wave numbers ν (cm⁻¹)/signal allocation of S-nitro ACC:
   3341 N—H stretch, amide group
   2954-2853 C—H stretch, aliphatic sp³-C—H species
   1736 C═O stretch, carboxyl group
   1661 C═O stretch, amide group
   1495-1376 asymmetric N—O stretch, nitro group
   1240-1186 symmetrische N—O stretch, nitro group
   1027 C—N stretch, amino acid.

### 5) Nitration of Amines

Treatment of urotropin with conc. nitric acid leads to perhydro-1,3,5-trinitro-1,3,5-triazin as major product. Synthesis of this species is therefore of technical interest.

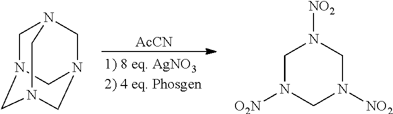

Urotropin is dissolved in acetonitril together with 8 equivalents of silver nitrate and is cooled to 0° C. in an ice bath. Phosgene (4 equivalents) is added dropwise and the reaction mixture is allowed to warm up to room temperature. Stirring is continued for one further hour. Afterwards silver salts are filtered off, ethyl acetate is added to the filtrate and the organic phase is washed three times with water. After separation and drying over sodium sulfate any volatiles are evaporated in vacuo. Crude products are taken up in acetone and the desired species was crystallized through cooling in a refrigerator.

### LITERATUR

[1] J. E. Gordon et al., J. Chromatog. 1970, 48, 532-534.
[2] J. E. Gordon et al., J. Org. Chem. 1970, 35(8), 2722-2725
[3] G. A. Olah, A. P. Fung, S. C. Narang, J. A. Olah, J. Org. Chem. 1981, 46(17), 3533-3537.
[4] G. K. Surya Prakash, C. Panja, T. Mathew, V. Surampudi, N. A. Petasis, G. A. Olah, Org. Lett. 2004, 6(13), 2205-2207.
[5] A. Khalafi-Nezhad et al., Helv. Chim. Acta 1984, 67, 906-915.
[6] G. A. Olah et al., J. Org. Chem. 1990, 55(17), 5179-5180.
[7] H. Burton, P. F. G. Praill, J. Chem. Soc. 1955, 729-731.
[8] E. Santaniello, M. Ravasi, P. Ferraboschi, J. Org. Chem. 1983, 48, 739-740.
[9] J. A. R. Rodrigues, A. P. O. Filho, P. J. S. Moran, Synth. Comm. 1999, 29(12), 2169-2174.
[10] F. Francis et al., Berichte 1906, 39, 3798-3804.
[11] M. E. Kurz, E. P. Zahora, D. Layman, J. Org. Chem. 1973, 38(13), 2277-2281.
[12] M. E. Kurz, E. Woodby, J. Org. Chem. 1976, 41(14).

What is claimed is:

1. A process for the conversion of hydroxyl, thiol, and amino groups into their nitric acid esters (nitrates: —ONO₂, —SNO₂ and —NRNO₂) by employing inorganic or organic nitric acid salts combined with a carbonic acid di- or monohalo derivative (oxidation number+IV) or the corresponding thiocarbonic acid analog.

2. A process as described in claim 1 where the cation of the nitric acid salt forms a insoluble or poorly soluble salt with the halogenide of the carbonic acid species in the applied solvent.

3. A process as described in claim 2 where the applied nitric acid salt is an inorganic metal nitric acid salt of the composition M^{x+}(NO₃⁻)ₓ.

4. A process as described in claim 3 where the applied nitric acid salt is silver nitrate (AgNO₃).

5. A process as described in claim 4 where the applied carbonic acid halogenide bears one or two chlorine atoms.

6. A process as described in claim 5 where the applied carbonic acid species is phosgene, diphosgene, triphosgene, thiophosgene, any chloroformic acid ester and any chlorothioformic acid ester.

7. A process as described in claim 6 where the applied carbonic acid species is phosgene and diphosgene.

8. A process as described in claim 2 where the applied generic carbonic acid species together with the nitric acid salt forms a mixed mono- or dianhydride of carbonic acid or thiocarbonic acid and nitric acid.

9. A process as described in claim 8 where the formation of said mixed anhydride is carried out in situ.

10. A process as described in claim 9 where all or a part of the participating reactants are combined in a single vessel

11. A process as described in claim 10 where the mixed anhydride species of carbonic acid or thiocarbonic acid spontaneously decomposes delivering gaseous small molecules and a nitronium species.

12. A process as described in claim 11 where the driving force of spontaneous decomposition is the system's increase in entropy.

13. A process as described in claim 11 where the decomposition of carbonic acid dinitrate in organic media delivers the required electrophilic nitronium species.

14. A process as described in claims 7 and 13 where acetonitrile is used as solvent.

15. A process as described in claim 14 to solely and selectively achieve nitration of hydroxyl, thiol and amino groups in the presence of aromatic and/or unsaturated functions.

16. A process as described in claim 14 to solely and selectively achieve nitration of hydroxyl, thiol and amino groups in the intramolecular presence of aromatic and/or unsaturated functions.

17. Polythiols, polyalcohols, polyamines and polyamides in varying degrees of nitration conserving their molecular and/or macromolecular structure where chain length, degree of conjunction and substitution does not deviate more than 2% from the original state of the starting materials.

18. A substance as described in claim 17 where the nitro compound is a (N-nitro)-alkylamino polymer or dendrimer with variable degree of nitration.

19. A substance as described in claim 18 where the nitro compound is a (N-nitro)-polyethylenimine in variable degree of nitration and polarity.
A substance as described in claim 18 where the nitro compound is an uncharged (N-nitro)-polyethylenimine in variable degree of nitration and polarity.
A substance as described in claim 18 where the nitro compound is an ionic (N-nitro)-polyethylenimine in variable degree of nitration and polarity.

20. A Nitromono-, nitrooligo-, or nitropolysaccharide in variable degree of nitration conserving their molecular and/or macromolecular structure where chain length, degree of conjunction and substitution does not deviate more than 2% from the original state of the starting materials.

21. A nitropolysaccharide as described in claim 20 with improved or altered material characteristics with regards to thermal stability and thermoplasticity.

22. A nitropolysaccharide as described in claim 21 based on starch, cellulose, chitin and chitosan.
A nitropolysaccharide as described in claim 21 which is a form of collodium (nominal dinitrate per monomer, degree of nitration˜11.11-12.5%),
A nitropolysaccharide as described in claim 21 which is a form of collodium (nominal dinitrate per monomer, degree of nitration˜11.11-12.5%), melting above T>165° C.,
A nitropolysaccharide as described in claim 21 which is a form of collodium (nominal dinitrate per monomer, degree of nitration˜11.11-12.5%), melting above T>170° C.,
A nitropolysaccharide as described in claim 21 which is a form of collodium (nominal dinitrate per monomer, degree of nitration˜11.11-12.5%), melting above T>175° C.,
A nitropolysaccharide as described in claim 21 which is a form of collodium (nominal dinitrate per monomer, degree of nitration˜11.11-12.5%), melting above T>180° C.,
A nitropolysaccharide as described in claim 21 which is a form of collodium (nominal dinitrate per monomer, degree of nitration˜11.11-12.5%), melting above T>182° C.

23. Nitromono- and nitrooligosaccharides in variable degrees of nitration which can be prepared from unprotected reducing sugars.

24. Nitromono- and nitrooligosaccharides as described in claim 23 which can be made from partially arylated reducing sugars without nitration of the aryl residue. Polynitrated derivatives of D-glucopyranose exceeding a nitrogen carbon ratio (% N:% C) of 0.4, α- and β-anomers of 1,2,3,4,6-O-pentanitro-D-glucopyranoside (pernitro glucose), methyl-4,6-O-benzylidene-2,3-O-dinitro-α-D -glucopyranoside, methyl-4,6-O-benzy-lidene-2-O-nitro-α-D-glucopyrano side and methyl-4,6-O-benzylidene-3-O-nitro-α-D-glucopyranoside as possible building blocks for synthesis or synthesis auxiliary.
